(19) United States
(12) Patent Application Publication
(10) Pub. No.: US 2026/0057986 A1
(43) Pub. Date: Feb. 26, 2026

(54) MULTI-PARAMETER GROUP BIOFEEDBACK TRAINING METHOD, APPARATUS, AND SYSTEM, AND STORAGE MEDIUM

(71) Applicants: KINGFAR INTERNATIONAL INC., Beijing (CN); Nanjing Kingfar Health Technology Inc., Nanjing (CN)

(72) Inventors: Qichao ZHAO, Beijing (CN); Ran YANG, Beijing (CN)

(21) Appl. No.: 19/375,263

(22) Filed: Oct. 31, 2025

Related U.S. Application Data
(63) Continuation of application No. PCT/CN2024/131531, filed on Nov. 12, 2024.

(30) Foreign Application Priority Data
Dec. 28, 2023 (CN) 202311852633.0

(51) Int. Cl. G16H 20/00

(52) U.S. Cl. CPC G16H 20/00

(57) ABSTRACT

Provided are a multi-parameter group biofeedback training method, apparatus, and system, and a storage medium. The method includes: receiving a predetermined training program and a predetermined training prescription that are set in a training management module, the training program being a combination of individual trainings, and the training prescription being a combination of long-term, time-sequenced phased trainings or training programs; and presenting the training scenario to a subject based on the training or the training prescription to execute the training, and collecting in real-time physiological data and/or target indicator data selected for the training. The training prescription includes predetermined training programs for a plurality of phases and a transition rule between the training programs for the plurality of phases. The training program includes one or more training tasks in a predetermined execution sequence. Each training task has a corresponding training scenario.

### CROSS-REFERENCE TO RELATED APPLICATIONS

This application is a continuation of International Application No. PCT/CN2024/131531 filed on Nov. 12, 2024, which claims priority to Chinese Patent Application No. 202311852633.0, titled “MULTI-PARAMETER GROUP BIOFEEDBACK TRAINING METHOD, APPARATUS, AND SYSTEM, AND STORAGE MEDIUM” and filed on Dec. 28, 2023, both of which are incorporated herein by reference in their entireties.

### FIELD

The present disclosure relates to the technical field of group biofeedback training in medical health, and particularly, to a multi-parameter group biofeedback training method, apparatus, and system, and a storage medium.

### BACKGROUND

Biofeedback training is a method that monitors and measures physiological indicators of the human body, feeds back information about these indicators to the individual, and enables the individual to self-regulate and train to control their physiological state. In the biofeedback training, commonly used physiological indicators include heart rate, electrocardiogram, skin conductance response, respiratory rate, and muscle electrical activity. These indicators can be collected and measured in real-time through sensors or devices. Through real-time feedback and generation of detection reports, different training methods are set based on individual detection results, which allows each person to monitor their own physical state and change these physiological indicators through regulation and training, achieving a purpose of improving personal health and controlling bodily responses.

Group biofeedback training is a training method that applies biofeedback training to a group environment involving multiple participants. In the group biofeedback training, the multiple individuals simultaneously receive feedback about their physical states and achieve their goals through interaction and collaboration. In multi-participant biofeedback training, individuals can encourage and motivate each other and promptly recognize differences, achieving better training outcomes. However, conventional biofeedback systems only support single-user operation and lack functionality for real-time communication among multiple users. These conventional biofeedback systems also lack training strategies or interaction modes for group collaboration. There is even an absence of objective evaluation standards; instead, feedback training results are subjectively assessed by relevant medical practitioners. Actual effectiveness of the training and rationality of the training cannot be quantified. Training is then repeated multiple times based on the assessment results, lacking standardized training procedures.

### SUMMARY

In view of this, embodiments of the present disclosure provide a multi-parameter group biofeedback training method and system, and a storage medium, to eliminate or overcome one or more defects existing in the related art.

In an aspect of the present disclosure, a multi-parameter group biofeedback training method is provided. The method is implemented based on a multi-parameter group biofeedback training system including a training management module. The method includes: obtaining a training program and a training prescription that are predetermined, in which each of the training program and the training prescription is configured in training management, and the training management module is configured to include a training scenario, the training program being a combination of individual trainings, and the training prescription being a combination of long-term, time-sequenced phased trainings or training programs; and presenting the training scenario to a subject based on the training or the training prescription to execute the training, and collecting in real-time physiological data and/or target indicator data selected for the training. The training prescription includes predetermined training programs for a plurality of phases and a transition rule between the training programs for the plurality of phases. The training program includes one or more training tasks that are predetermined to be executed, each of the one or more training tasks having a corresponding training scenario.

In some embodiments of the present disclosure, the method further includes, prior to receiving a predetermined training configuration set in the training management module: obtaining an assessment report and/or a training tool configured in the training management; and configuring a corresponding training program based on the assessment report and/or the training tool of the subject.

In some embodiments of the present disclosure, the multi-parameter group biofeedback training system includes: a user management module configured to manage training configurations and training records of different subjects. The user management module is configured to support operations on the training configuration and the training record of the subject, the operations including adding/deleting/modifying/querying, importing/exporting, batch importing, or batch deletion.

In some embodiments of the present disclosure, the multi-parameter group biofeedback training system further includes: a group management module configured to perform group management on different subjects based on a set need. The group management module is configured to support the operations on the training configuration and the training record of the subject, the operations including adding/deleting/modifying/querying, importing/exporting, batch importing, or batch deletion.

In some embodiments of the present disclosure, the training management module is configured to support custom configuring a training configuration. The custom configuring the training configuration includes: selecting target indicator data based on multi-parameter group biofeedback training; selecting a training scenario compatible with the multi-parameter group biofeedback training, types of the training scenario including any one or a combination of a VR scenario, an audio-video scenario, a virtual multi-person interactive animation scenario, a virtual multi-person interactive game scenario, a multi-person interactive virtual-real fusion scenario, a screen-based 3D scenario, a visualization chart component, or an audio-video game resource scenario; and performing an interactive setting between different scenarios based on a transition rule between different training programs in the training prescription that is pre-obtained. The interactive setting includes an ending manner setting for ending the training prescription. The transition rule includes a transition condition setting for transitioning between training programs and a transition relationship network.

In some embodiments of the present disclosure, the presenting the training scenario to the subject based on the training or the training prescription to execute the training, and collecting in real-time the physiological data and/or the target indicator data selected for the training includes: in a physiological feedback training scenario, selecting the target indicator data by freely combining physiological channels.

In some embodiments of the present disclosure, the method further includes: generating a group biofeedback training report based on a target indicator data presentation manner pre-configured by the training management module. The target indicator data presentation manner includes target indicator data to be presented and a visualization chart. The custom configuring the training configuration further includes: configuring the target indicator data presentation manner. The visualization chart includes, but is not limited to, one or more of a pie chart, a bar chart, or a Venn diagram.

In some embodiments of the present disclosure, the training management module further includes a pre-built-in calculation formula, a classification algorithm model, and a recommendation algorithm model. The custom configuring the training configuration further includes: calculating a group biofeedback training result based on the target indicator data using the built-in calculation formula; classifying, based on the group biofeedback training result, subjects in accordance with a predetermined classification rule using the classification algorithm model; and recommending, based on the group biofeedback training result, a training program of a next phase to the subject in accordance with a predetermined recommendation rule using the recommendation algorithm model.

In some embodiments of the present disclosure, different phases of the training prescription include at least two items of an assessment phase, a training phase, a validation phase, or an application phase. Different training phases include corresponding training programs. The training prescription includes a transition rule that is pre-configured between different training programs and a recommendation rule.

In some embodiments of the present disclosure, multi-parameter group biofeedback training is performed by the multi-parameter group biofeedback training system through a process management approach.

In some embodiments of the present disclosure, the method further includes: subsequent to obtaining the target indicator data, performing a data analysis and generating a data profile based on the obtained target indicator data, custom building a user ability assessment and training model, swiftly establishing a data model and a data profile system through a drag-and-drop operation to build an ability model and/or a data profile, obtaining the target indicator data subsequent to a completion of group biofeedback training for the subject, and obtaining an ability model and/or a data profile of the subject based on the data model and the data profile system.

In another aspect of the present disclosure, a multi-parameter group biofeedback training apparatus is provided. The apparatus is configured to implement the steps of the method according to any of the above embodiments.

In yet another aspect of the present disclosure, a multi-parameter group biofeedback training system is provided. The multi-parameter group biofeedback training system includes: a processor; and a memory having computer instructions stored thereon. The processor is configured to execute the computer instructions stored in the memory. When the computer instructions are executed by the processor, the multi-parameter group biofeedback training system implements the steps of the method according to any of the above embodiments.

In still yet another aspect of the present disclosure, a computer-readable storage medium is provided. The computer-readable storage medium stores a computer program. The computer program, when executed by a processor, implements the steps of the method according to any of the above embodiments.

With the multi-parameter group biofeedback training method and system, and the storage medium provided by the present disclosure, the subject can be trained based on the training configuration that is pre-selected. By incorporating the transition rule between the training programs within the training prescription, multi-mode training for the subject is achieved, enabling standardized multi-parameter group biofeedback training.

Additional advantages, objectives, and features of the present disclosure will be set forth in part in the following description, and will in part become apparent to those skilled in the art upon study of the following contents or be learned from practicing of the present disclosure. The objectives and other advantages of the present disclosure will be realized and attained by the structure particularly pointed out in the description and the accompanying drawings.

Those skilled in the art will appreciate that the objectives and advantages that can be achieved with the present disclosure are not limited to those specifically set forth above, and that the above and other objectives that can be achieved by the present disclosure will become clearer from the following detailed description.

### BRIEF DESCRIPTION OF THE DRAWINGS

The accompanying drawings described here are used to provide a further understanding of the present disclosure and constitute a part of the present disclosure, and do not constitute a limitation on the present disclosure.

FIG. 1 is a flowchart of a multi-parameter group biofeedback training method according to an embodiment of the present disclosure.

FIG. 2 is a flowchart of custom configuring a training configuration according to an embodiment of the present disclosure.

FIG. 3 is an example of transitions between training programs according to an embodiment of the present disclosure.

### DETAILED DESCRIPTION

In order to make the objectives, technical solutions, and advantages of the present disclosure more clearly understood, the present disclosure is further described in detail below with reference to the embodiments and the accompanying drawings. Exemplary embodiments of the present disclosure and descriptions thereof are intended to explain the present disclosure, and do not constitute a limitation on the present disclosure.

It should be further noted that, in order to avoid obscuring the present disclosure due to unnecessary details, only structures and/or processing steps closely related to the solutions according to the present disclosure are illustrated in the accompanying drawings, while other details that are not closely related to the present disclosure are omitted.

It should be emphasized that the terms “comprise/contain”, when used in this specification, specify the presence of features, elements, steps, or components, but do not preclude the presence or addition of one or more other features, elements, steps, or components.

It should also be noted that, unless otherwise specified, the term “connection” may be used herein to refer not only to a direct connection but also to an indirect connection through an intermediate.

Hereinafter, the embodiments of the present disclosure will be described with reference to the accompanying drawings. In the accompanying drawings, same or similar components or same or similar steps are denoted by same reference numerals.

To solve problems existing in conventional human factor testing and training methods, the present disclosure provides a multi-parameter group biofeedback training method and system, and a storage medium.

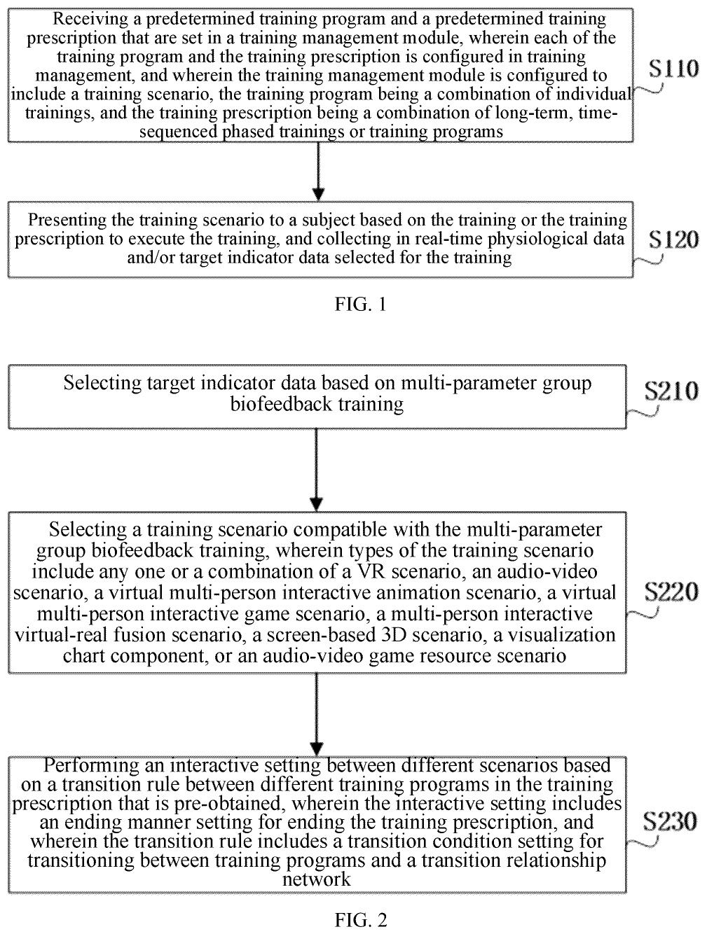

FIG. 1 is a flowchart of a multi-parameter group biofeedback training method according to an embodiment of the present disclosure. The method is implemented based on a multi-parameter group biofeedback training system including a training management module. The method includes operations at blocks.

At block S110, a training program and a training prescription that are predetermined are obtained. Each of the training program and the training prescription is configured in training management. The training management module is configured to include a training scenario. The training program is a combination of individual trainings. The training prescription is a combination of long-term, time-sequenced phased trainings or training programs.

At block S120, the training scenario is presented to a subject based on the training or the training prescription to execute the training, and physiological data and/or target indicator data selected for the training are collected in real-time.

The training prescription includes predetermined training programs for a plurality of phases and a transition rule between the training programs for the plurality of phases. The training program includes one or more training tasks that are predetermined to be executed. Each of the one or more training tasks has a corresponding training scenario.

By adopting the embodiment of the present disclosure, the subject can be trained based on the training configuration that is pre-selected. By incorporating the transition rule between the training programs within the training prescription, multi-mode training for the subject is achieved, enabling standardized multi-parameter group biofeedback training and achieving high-freedom multi-channel biofeedback training.

In some embodiments of the present disclosure, the method further includes, prior to receiving a predetermined training configuration set in the training management module: obtaining an assessment report and/or a training tool configured in the training management; and configuring a corresponding training program based on the assessment report and/or the training tool of the subject. By adopting the embodiments of the present disclosure, the corresponding training program can be configured based on a detection report of the subject, enabling targeted training for the subject under different circumstances.

The configuration in the training management module includes training, program, and prescription as three entity objects. The training is a configured tool for assessment or training, like a scale. The program is a combination of trainings, a concept of a training package. The prescription is a phased combination of training and program and contains a timeline, allowing for a configuration of a complete training course over a period of time.

In an embodiment of the present disclosure, the multi-parameter group biofeedback training system includes: a user management module configured to manage training configurations and training records of different subjects. The user management module is configured to support operations on the training configuration and the training record of the subject, the operations including adding/deleting/modifying/querying, importing/exporting, batch importing, or batch deletion.

By adopting the embodiment of the present disclosure, personnel undergoing group biofeedback training can be managed, avoiding confusion when there are many users participating in the training.

Further, in some embodiments of the present disclosure, the multi-parameter group biofeedback training system further includes: a group management module configured to perform group management on different subjects based on a set need. The group management module is configured to support the operations on the training configuration and the training record of the subject, the operations including adding/deleting/modifying/querying, importing/exporting, batch importing, or batch deletion.

By adopting the embodiments of the present disclosure, subjects (users) can be managed in groups, orderly managing the subjects participating in the group biofeedback training.

FIG. 2 is a flowchart of custom configuring a training configuration according to an embodiment of the present disclosure. In some embodiments of the present disclosure, the training management module is configured to support custom configuring a training configuration. Custom configuring the training configuration includes operations at blocks.

At block S210, target indicator data is selected based on multi-parameter group biofeedback training.

At block S220, a training scenario compatible with the multi-parameter group biofeedback training is selected. Types of the training scenario include any one or a combination of a VR scenario, an audio-video scenario, a virtual multi-person interactive animation scenario, a virtual multi-person interactive game scenario, a multi-person interactive virtual-real fusion scenario, a screen-based 3D scenario, a visualization chart component, or an audio-video game resource scenario.

At block S230, an interactive setting is performed between different scenarios based on a transition rule between different training programs in the training prescription that is pre-obtained. The interactive setting includes an ending manner setting for ending the training prescription. The transition rule includes a transition condition setting for transitioning between training programs and a transition relationship network.

By adopting the embodiment of the present disclosure, the training configuration to be selected in the training management module can be configured, achieving multi-mode and flexible group biofeedback training. According to the embodiment of the present disclosure, needs of different patients can also be satisfied, time spent by medical staff on repetitive configurations can be reduced, and standardization and normalization of an assessment and treatment system can be ensured. For example, for patients who need rapid recovery, they may directly go to a training center and choose one training or two trainings; for patients who need long-term treatment, they may first undergo an assessment, then receive a prescription, and proceed with complete follow-up treatment; and physical and mental states of the patient can be comprehensively assessed in combination with a program, such as using neurofeedback to assess the nervous system of the patient and using HRV training to assess the autonomic nervous system of the patient.

Biofeedback training has very broad applications in the field of medical health. It is a treatment method based on a physiological collection device. Through biofeedback technology, information about physiological changes that are not easily perceived by individuals is collected and amplified, and then displayed in an easily recognizable visual or auditory form. After perceiving these physiological or pathological changes, individuals perform conscious control and psychological training to control and regulate abnormal physiological responses, achieving purposes of adjusting bodily functions and preventing and treating diseases.

Feedback information is collected in the present disclosure. Human biological signals are obtained through a biological collection device, which include, but are not limited to, electroencephalography information, brain imaging information, heart rate, electrocardiogram, galvanic skin response, electromyography (muscle electrical signals), blood pressure and blood oxygen, eye movement, and other physiological and sensory signals. By analyzing collected signals from different dimensions, feedback report corresponding to each individual is generated. Individuals conduct symptom-targeted training based on the feedback reports, and feedback reports from the same or different fields can be integrated to form group training.

Biofeedback technology can be applied to various psychosomatic diseases related to stress, such as tension headaches, gastric ulcers, and chronic anxiety. In addition, biofeedback technology is widely used in fields such as neurological rehabilitation, sports rehabilitation, and psychological rehabilitation. For example, neurofeedback technology monitors and trains brainwave activity of an individual to help regulate and improve brain function, and is widely used in neurological rehabilitation fields such as stroke rehabilitation, treatment of attention deficit hyperactivity disorder, and movement control disorders. Heart rate variability feedback technology monitors heart rate variability of an individual to help improve cardiovascular function and regulate the autonomic nervous system, and is widely used in fields such as stress management, treatment of anxiety disorders, and improvement of mental health.

Neurofeedback technology can assist in regulating and improving brain function, and is widely used in the neurological rehabilitation fields such as stroke rehabilitation, treatment of attention deficit hyperactivity disorder, and movement control disorders. Individuals enhance their awareness of their own brain states by observing brainwave graphs and specific visualized feedback, achieving self-regulation and recovery of brain function.

In addition, heart rate variability feedback technology is widely used in the fields such as stress management, treatment of anxiety disorders, and improvement of mental health. Individuals improve their perception of their own cardiovascular state by observing heart rate variability graphs and performing specific breathing exercises, and enhance heart rate variability by regulating respiratory rate and depth, achieving self-regulation and improvement of cardiac health.

Muscle electromyographic signal feedback technology monitors muscle electrical activity of an individual to help improve muscle control and movement function, and is extensively applied in fields such as rehabilitation from muscle denervation injuries, pain management, and enhancement of sports skills.

Regarding a treatment scheme of a biofeedback training system based on electroencephalogram signal collection, when an individual with anxiety disorders exhibits symptoms such as excessive tension, worry, and fear, electroencephalogram biosignals are collected and analyzed to determine a degree of tension, worry, or fear of the individual, and feedback on a state of the individual is provided to assist in training and improvement. Under normal circumstances, standardized testing protocols are used to determine a parameter range, which is then adjusted through multiple trainings. In addition, a timely detection is performed to conduct a comparison with a previous state parameter, which allows a patient to promptly understand a symptom improvement. In this way, real-time dynamic monitoring and early warning of electroencephalogram changes are realized, achieving an effect of treating anxiety disorders.

Further, an electroencephalogram biofeedback training system can upload electroencephalogram waveforms and signals to a computer end or a cloud platform using electroencephalogram biofeedback technology, and then conduct a series of dynamic electroencephalogram change monitoring to determine type, severity, and specific conditions of insomnia. Training can then be repeated according to instructions of a doctor to alleviate symptoms. Additionally, patients with similar experiences can form a group biofeedback training model, for supervising each other, sharing methods to promote mutual improvement, and improving treatment schemes.

In some embodiments of the present disclosure, presenting the training scenario to the subject based on the training or the training prescription to execute the training (multi-parameter group biofeedback training), and collecting in real-time the physiological data and/or the target indicator data selected for the training includes: in a physiological feedback training scenario, selecting the target indicator data by freely combining physiological channels.

By adopting the embodiments of the present disclosure, physiological feedback training can be performed within the physiological feedback training scenario. The group biofeedback training includes physiological feedback training.

In some embodiments of the present disclosure, the method further includes, subsequent to collecting in real-time the target indicator data: generating a group biofeedback training report based on a target indicator data presentation manner pre-configured by the training management module. The target indicator data presentation manner includes target indicator data to be presented and a visualization chart. In addition, compatible with this, custom configuring the training configuration further includes configuring the target indicator data presentation manner. The visualization chart includes, but is not limited to, one or more of a pie chart, a bar chart, or a Venn diagram. The present disclosure is not limited in this regard. The target indicator data presentation manner is not limited to those listed. Those skilled in the art can freely extend the target indicator data presentation manner.

By adopting the embodiments of the present disclosure, the group biofeedback training report can be generated based on the obtained target indicator data, visually presenting the training result to the subject or a third-party person.

In some embodiments of the present disclosure, the training management module further includes a pre-built-in calculation formula, a classification algorithm model, and a recommendation algorithm model. Custom configuring the training configuration further includes: (1) calculating a group biofeedback training result based on the target indicator data using the built-in calculation formula; (2) classifying, based on the group biofeedback training result, subjects in accordance with a predetermined classification rule using the classification algorithm model; and (3) recommending, based on the group biofeedback training result, a training program of a next phase to the subject in accordance with a predetermined recommendation rule using the recommendation algorithm model.

By adopting the embodiments of the present disclosure, the transition rule between different training programs in the training prescription are enriched, enabling more flexible group biofeedback training.

In some embodiments of the present disclosure, different phases of the training prescription include at least two of an assessment phase, a training phase, a validation phase, or an application phase; different training phases include corresponding training programs; and the training prescription includes a transition rule that is pre-configured between different training programs and a recommendation rule.

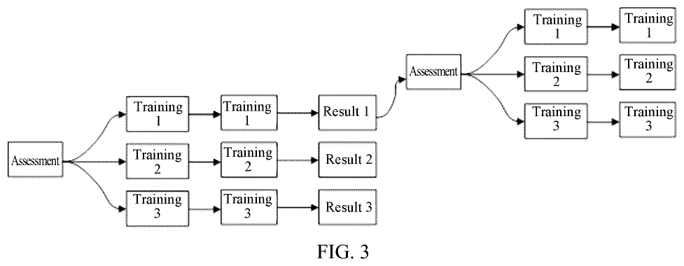

FIG. 3 is an example of transitions between training programs according to an embodiment of the present disclosure. The group biofeedback training result is calculated. Based on the group biofeedback training result, the training program for the corresponding phase is arranged. The training program includes a plurality of training tasks. As illustrated in FIG. 3, the training program includes two consecutive Trainings 1, two consecutive Trainings 2, or two consecutive Trainings 3. An assessment result can be calculated based on Result 1. A next phase of training can proceed.

In a specific embodiment of the present disclosure, the training program supports a custom combination of multiple training tasks (refer to Training 1, Training 2, and Training 3 in FIG. 3). The training program also supports selecting contents such as built-in scales, questionnaires, cognitive assessments, cognitive trainings, or behavioral experiments provided by the system, forming a comprehensive multi-dimensional, multi-type training program. An advanced mode of the program supports building a complete training program model based on the training result, i.e., adding a conditional transition function within the training program, enabling transitions between training tasks. As illustrated in FIG. 3, a transition logic between training tasks can be configured, and a configuration of transition rules and transition trainings based on the training results can be supported, which facilitates generation of personalized training programs for users at different levels.

In some embodiments of the present disclosure, multi-parameter group biofeedback training is performed by the multi-parameter group biofeedback training system through a process management approach. The user can directly select the above training prescription for training, or an administrator can assign trainings uniformly. A process is created by the administrator. The administrator can freely combine the above trainings and select to assign the trainings to specific groups, individuals, or multiple users. After the process is successfully created, a user progress can be tracked and monitored, or overall control of group training can be achieved. The administrator can adjust the training program or the training progress at any time based on a user result and feedback.

In some embodiments of the present disclosure, the method further includes: subsequent to obtaining the target indicator data, performing a data analysis and generating a data profile based on the obtained target indicator data, custom building a user ability assessment and training model, swiftly establishing a data model and a data profile system through a drag-and-drop operation to build an ability model and/or a data profile, obtaining the target indicator data subsequent to a completion of group biofeedback training for the subject, and obtaining an ability model and/or a data profile of the subject based on the data model and the data profile system. Subsequent to a completion of the training, generating a data profile of the subject based on an algorithm and a classification rule that are pre-stored in a training configuration, and updating in real-time an assessment report of the subject that has completed the training. The assessment report at least includes the physiological data and/or the target indicator data of the subject.

By adopting the embodiments of the present disclosure, a result of the group biofeedback training for the subject (user) can be analyzed through data analysis and data profile, generating a personalized ability model and/or data profile for the subject.

Further, in a specific embodiment of the present disclosure, a data analysis and data mining are performed with granularity of each data field and data metric result using data analysis technology. Custom building the user ability assessment and the training model is supported. The data model and the data profile system are swiftly established through the drag-and-drop operation. For example, a physical and mental assessment model for the user is built. A data source, a primary indicator, a secondary indicator, and the like for the model are selected. A calculation formula for each level is configured. The formula supports calculation methods such as basic operations, function operations, standard score calculations, normalization processing, and/or deep learning algorithms. In this way, a user profile or an ability model is built. After the training or the assessment for the user is completed, data is generated, and thus the user can view his/her own ability model and data profile. In addition, the system further provides a plurality of physical and mental state early warning and state recognition algorithms that can calculate physical and mental states of the user in real-time based on physical and mental data, training data, and basic data of the user, and performs classification or issues early warnings. The system further provides a custom algorithm extension function, allowing the user to upload an open-source algorithm or a self-developed algorithm as needed for training or modeling.

The data profile refers to a holistic, multi-dimensional model of the user or the entity that is formed by abstracting, processing, and integrating information such as attributes, characteristics, and behaviors of the user or entity through an analysis of massive amounts of data. A purpose of data profile is to better understand the user or the entity, providing the user or the entity with higher quality services or products. The data profile can be applied in various scenarios, for example: (1) user profile: used to understand attributes, interests, needs, etc., of the user, for providing personalized recommendations, precision marketing, and other services to the user; (2) product profile: used to understand product usage, user feedback, etc., improving product design and enhancing use experience; and (3) enterprise profile: used to understand enterprise scale, industry, customers, etc., formulating strategies more suitable for enterprise development.

For example, for people in certain industries, ability-based selections need to be set, which require continuous testing, feedback, and training within a group to achieve a purpose of selection. For example, pilots need to possess excellent vision and hearing to accurately identify and determine various situations during flight. Test results of vision and hearing can serve as important indicators for assessing physical fitness of candidates. Therefore, brainwave and brain imaging information that characterize vision and hearing can be collected from physiological parameters of the pilots, and a standard range for an optimal state can be identified and determined. Then, based on set paradigms for an enhancement of abilities such as vision and hearing, continuous training can be conducted to meet requirements for qualified pilots.

For a group of pilots who need to possess high levels of strength and endurance to maintain abundant physical strength and endurance during long-duration, high-intensity flights, the group biofeedback training method can provide feedback training on strength and endurance, including muscle strength tests, grip strength tests, aerobic endurance tests, etc.

Of course, the biofeedback system provided in the present disclosure is not limited to the field of pilots. The biofeedback system can be applied in other fields or by other people.

In a specific embodiment of the present disclosure, the training management module is configured to manage individual training resources. The training resource includes the training prescription, including classifications such as assessment, training, and group training, and support a custom classification. During a custom configuration in the training management module, the following steps are included.

(1) In a biofeedback training process, training is conducted based on the biofeedback system. Physiological channels can be freely combined. A physiological indicator and a visualization chart that need to be fed back to the user can be selected. \textcircled{1} A presentation form of an indicator result includes a numerical form, a dashboard, and a score bar. \textcircled{2} A form of the visualization chart includes a line chart, a bar chart, a spectrogram, and a pie chart. \textcircled{3} The system performs an intelligent layout based on the selected indicator and chart. An intelligent layout engine can calculate an optimal layout based on the indicator, and the size and the quantity of charts, which is beneficial for feedback on physiological, physical and mental states during the training.

### (2) Free Selection of the Training Scenario Used for Human Factor Testing Training

\textcircled{1} Scenario types include a VR scenario, an audio-video scenario, a multi-person interactive animation scenario, a multi-person interactive game scenario, etc. The system can have built-in scenario resources of the above types. The training scenario includes a dynamic multi-person interactive virtual-real fusion scenario, a screen-based 3D scenario, a visualization chart component, and an audio-video game resource scenario, and supports development of the above scenario resources through an expansion. These scenario resources are used to configure training. The user can select the above scenarios types as needed, or custom upload a file such as an audio-video material and an animation. Biofeedback training, relaxation training, and other human factor testing trainings can be conducted by displaying the above scenarios.

\textcircled{2} All scenarios developed by the system are dynamic. After the scenario is selected, the interactive setting can be performed with the scenario. For example, in the VR scenario developed by the system, an input indicator can be set. Elements such as images, colors, and motion effects of the scenario change based on the numerical change of the input indicator. A multi-person interactive scenario supports intelligent grouping of multiple people. During the training, cooperation or competitive games can be conducted based on changes in physiological indicator values within and between groups. A game scenario displays a dynamic result in real-time and provides feedback to individuals and groups.

### (3) Other Configurations (Including the Built-In Formula, Classification Algorithm Model, and Recommendation Algorithm Model)

\textcircled{1} The system provides a default training report and supports the custom configuration under the advanced mode. Report content includes basic training information, basic personnel information, a visualized indicator result and a process recording chart, result interpretation, a guidance suggestion, and an intelligent recommendation. Under an advanced configuration mode, the indicator result and the visualization chart that are presented in the report can be configured. The chart can be presented in different formats, such as pie chart, bar chart, etc. The result interpretation supports a custom configuration of calculation rules. The built-in calculation formula of the system can be selected, or manual input of a formula is supported. Formula calculations based on algorithms such as mean values and standard scores are supported. Adding the result interpretation for different results are also supported. For example, a classification algorithm is used for classification, with results being high, medium, or low. Custom result interpretations and guidance suggestions can be defined for each level of users, which facilitates review of personalized report results and suggestions by the users after completing the training. In addition, extraction, matching, and training of a user feature and a characteristic is supported in combination with the recommendation algorithm, to provide intelligent recommendations for other trainings to the users. The recommendation rule can be customized or automatically generated based on the training result, the user feature, a training feature, etc.

In a specific embodiment of the present disclosure, a report center saves all reports, generates reports with the granularity of each user and each training. The report center supports generating a complete program, a prescription report, etc., based on the multi-mode training such as the training program and the training prescription. Also, the report center supports generating an individual training record or a group training record based on users or groups. Further, the report center supports generating a trend analysis report for longitudinal tracking and a comparison report for a horizontal comparison.

By using the built-in formula, the classification algorithm model, and the recommendation algorithm model according to the above embodiments of the present disclosure, training prescriptions for multi-mode human factor testing training can be provided more flexibly, for conducting targeted training on the subject to achieve an expected result.

\textcircled{2} Ending manner setting: one can choose to end by default time or stop upon a game completion, or one can customize an interactive way to end the training, such as selecting a button or a mouse operation, etc.

Corresponding to the above method, the present disclosure further provides a multi-parameter group biofeedback training system. The system includes a computer device. The computer device includes a processor and a memory. The memory stores computer instructions. The processor is configured to execute the computer instructions stored in the memory. When the computer instructions are executed by the processor, the system implements the steps of the method described above.

Specifically, the multi-parameter group biofeedback training system includes the user management module, the group management module, the training management module, a program management module, a prescription management module, a process management module, a report management module, and a data analysis and data profile module.

The user management module is configured to support the operations such as adding/deleting/modifying/querying, importing/exporting, batch importing, and batch deletion. The group management module is configured to form a group based on needs of group treatment. The group is a combination of multiple users who share the same treatment, testing, or training process or purpose. The group management module is functionally identical to the user management module.

The prescription management module is configured to manage training programs for different phases. The prescription contains multiple phases. Each phase supports custom selecting and combining the above training tasks or training programs to form a complete systematic and periodic training program. This prescription is not equivalent to a clinical prescription; rather, it is a conceptual summary of the above periodic and complete training cycles and programs. (1) The prescription can be custom-created with phases. Common phases include the assessment phase, the training phase, the validation phase, the application phase, etc., which can be combined as needed. (2) Training and program for each phase can be configured with a transition logic and a recommendation rule to form a training prescription. Personalized training paths and programs can be generated for different users.

It should be noted that, in a specific implementation process, the training task, the training program, and the training prescription are three entity objects. The training configuration is used to configure a single training task. The training task is a configured tool used for assessment or training, and recorded in a form of a “training resource,” similar to a “scale.” The training program is a combination of training tasks, similar to the concept of a training package. The training prescription is a phased combination of training and program and contains the timeline, allowing for the configuration of the complete training course over a period of time. Taking the group biofeedback training in the medical field as an example, benefits of this approach are that: the needs of different patients can be satisfied, the time spent by medical staff on the repetitive configurations can be reduced, and the standardization and the normalization of the assessment and treatment system can be ensured. For example, for patients who need rapid recovery, they may directly go to the training center and choose one training or two trainings; for patients who need long-term treatment, they may first undergo the assessment, then receive the prescription, and proceed with the complete follow-up treatment; and the physical and mental states of the patient can be comprehensively assessed in combination with the program, such as using the neurofeedback to assess the nervous system of the patient and using HRV training to assess the autonomic nervous system of the patient. Accordingly, at block S110, the predetermined training configuration, set in the training management module, of the training program and the training prescription is received. The training program includes a combination of one or more training tasks. The training management module includes a training scenario compatible with the training task. The training prescription includes a combination of long-term, time-sequenced phased training tasks and training programs. The training configuration includes the training scenario, the training prescription, and the target indicator data. The training prescription includes training programs for a plurality of phases.

According to embodiments of the present disclosure, a multi-parameter group biofeedback training apparatus is further provided. The apparatus is configured to implement the steps of the method according to any of the above embodiments.

According to embodiments of the present disclosure, a computer-readable storage medium is further provided. The computer-readable storage medium stores a computer program. The computer program, when executed by a processor, implements the steps of the above-described method. The computer-readable storage medium may be a tangible storage medium such as a Random Access Memory (RAM), a memory, a Read-Only Memory (ROM), an electrically programmable ROM, an electrically erasable programmable ROM, a register, a floppy disk, a hard disk, a removable storage disk, a CD-ROM, or any other form of storage medium known in the art.

With the multi-parameter group biofeedback training method and system, and the storage medium provided by the present disclosure, the subject can be trained based on the training configuration that is pre-selected. By incorporating the transition rule between the training programs within the training prescription, the multi-mode training for the subject is achieved, enabling the standardized multi-parameter group biofeedback training.

Those skilled in the art could be aware that, exemplary components, systems, and methods described in combination with embodiments disclosed herein may be implemented by hardware, software, or a combination thereof. Whether these functions are executed by hardware or software is dependent on particular use and design constraints of the technical solutions. Professionals may adopt different methods for different particular uses to implement described functions, which should not be regarded as going beyond the scope of the present disclosure. When implemented in hardware, elements of the present disclosure may be, for example, electronic circuits, Application Specific Integrated Circuits (ASICs), appropriate firmware, plug-ins, and function cards. When implemented in software, the elements of the present disclosure are programs or code segments used to execute required tasks. The programs or the code segments may be stored in a machine-readable medium or transmitted over a transmission medium or a communication link via a data signal carried in a carrier.

It should be understood that the present disclosure is not limited to specific configurations and processing described above and illustrated in the figures. For the sake of conciseness, a detailed description of known methods is omitted here. In the above-described embodiments, several specific steps are described and illustrated as examples. However, the process of the method of the present disclosure is not limited to the specific steps described and illustrated. Those skilled in the art can make various changes, modifications, and additions, or change the order of the steps after grasping the spirit of the present disclosure.

In the present disclosure, features described and/or illustrated for one embodiment may be used in the same manner or in a similar manner in one or more other embodiments, and/or may be combined with or substituted for features of other embodiments.

Although some embodiments of the present disclosure are described above, the present disclosure is not limited to these embodiments. For those skilled in the art, various changes and variations can be made to the embodiments of the present disclosure. Any modification, equivalent replacement, improvement, etc. made within the spirit and principle of the present disclosure shall fall within the protection scope of the present disclosure.

What is claimed is:

1. A multi-parameter group biofeedback training method, implemented based on a multi-parameter group biofeedback training system comprising a training management module, the method comprising:
obtaining a training program and a training prescription that are predetermined, wherein each of the training program and the training prescription is configured in training management, and wherein the training management module is configured to comprise a training scenario, the training program being a combination of individual trainings, and the training prescription being a combination of long-term, time-sequenced phased trainings or training programs; and
presenting the training scenario to a subject based on the training or the training prescription to execute the training, and collecting in real-time physiological data and/or target indicator data selected for the training, wherein:
the training prescription comprises predetermined training programs for a plurality of phases and a transition rule between the training programs for the plurality of phases; and
the training program comprises one or more training tasks that are predetermined to be executed, each of the one or more training tasks having a corresponding training scenario.

2. The method according to claim 1, further comprising, prior to receiving a predetermined training configuration set in the training management module:
obtaining an assessment report and/or a training tool configured in the training management; and
configuring a corresponding training program based on the assessment report and/or the training tool of the subject.

3. The method according to claim 1, wherein the multi-parameter group biofeedback training system comprises:
a user management module configured to manage training configurations and training records of different subjects, wherein the user management module is configured to support operations on the training configuration and the training record of the subject, the operations comprising adding/deleting/modifying/querying, importing/exporting, batch importing, or batch deletion.

4. The method according to claim 3, wherein the multi-parameter group biofeedback training system further comprises:
a group management module configured to perform group management on different subjects based on a set need, wherein the group management module is configured to support the operations on the training configuration and the training record of the subject, the operations comprising adding/deleting/modifying/querying, importing/exporting, batch importing, or batch deletion.

5. The method according to claim 1, wherein the training management module is configured to support custom configuring a training configuration, wherein said custom configuring the training configuration comprises:
selecting target indicator data based on multi-parameter group biofeedback training;
selecting a training scenario compatible with the multi-parameter group biofeedback training, wherein types of the training scenario comprise any one or a combination of a VR scenario, an audio-video scenario, a virtual multi-person interactive animation scenario, a virtual multi-person interactive game scenario, a multi-person interactive virtual-real fusion scenario, a screen-based 3D scenario, a visualization chart component, or an audio-video game resource scenario; and
performing an interactive setting between different scenarios based on a transition rule between different training programs in the training prescription that is pre-obtained, wherein the interactive setting comprises an ending manner setting for ending the training prescription, and wherein the transition rule comprises a transition condition setting for transitioning between training programs and a transition relationship network.

6. The method according to claim 5, wherein said presenting the training scenario to the subject based on the training or the training prescription to execute the training, and collecting in real-time the physiological data and/or the target indicator data selected for the training comprises:
in a physiological feedback training scenario, selecting the target indicator data by freely combining physiological channels.

7. The method according to claim 5, further comprising:
generating a group biofeedback training report based on a target indicator data presentation manner pre-configured by the training management module, wherein the target indicator data presentation manner comprises target indicator data to be presented and a visualization chart, wherein:
said custom configuring the training configuration further comprises:
configuring the target indicator data presentation manner, wherein the visualization chart comprises, but is not limited to, one or more of a pie chart, a bar chart, or a Venn diagram.

8. The method according to claim 5, wherein:
the training management module further comprises a pre-built-in calculation formula, a classification algorithm model, and a recommendation algorithm model; and
wherein said custom configuring the training configuration further comprises:
calculating a group biofeedback training result based on the target indicator data using the built-in calculation formula;
classifying, based on the group biofeedback training result, subjects in accordance with a predetermined classification rule using the classification algorithm model; and
recommending, based on the group biofeedback training result, a training program of a next phase to the subject in accordance with a predetermined recommendation rule using the recommendation algorithm model.

9. The method according to claim 1, further comprising:
subsequent to a completion of the training, generating a data profile of the subject based on an algorithm and a classification rule that are pre-stored in a training configuration, and updating in real-time an assessment report of the subject that has completed the training, wherein the assessment report at least comprises the physiological data and/or the target indicator data of the subject.

10. The method according to claim 1, wherein:
different phases of the training prescription comprise at least two of an assessment phase, a training phase, a validation phase, or an application phase;
different training phases comprise corresponding training programs; and
the training prescription comprises a transition rule that is pre-configured between different training programs and a recommendation rule.

11. The method according to claim 1, wherein multi-parameter group biofeedback training is performed by the multi-parameter group biofeedback training system through a process management approach.

12. The method according to claim 1, further comprising:
subsequent to obtaining the target indicator data, performing a data analysis and data profile based on the obtained target indicator data, custom building a user ability assessment and training model, swiftly establishing a data model and a data profile system through a drag-and-drop operation to build an ability model and/or a data profile, obtaining the target indicator data subsequent to a completion of group biofeedback training for the subject, and obtaining an ability model and/or a data profile of the subject based on the data model and the data profile system.

13. A multi-parameter group biofeedback training system, comprising:
a processor; and
a memory having computer instructions stored thereon, wherein:
the processor is configured to execute the computer instructions stored in the memory; and
when the computer instructions are executed by the processor, the multi-parameter group biofeedback training system implements a multi-parameter group biofeedback training method, the system comprising a training management module, and the method comprising:
obtaining a training program and a training prescription that are predetermined, wherein each of the training program and the training prescription is configured in training management, and wherein the training management module is configured to comprise a training scenario, the training program being a combination of individual trainings, and the training prescription being a combination of long-term, time-sequenced phased trainings or training programs; and
presenting the training scenario to a subject based on the training or the training prescription to execute the training, and collecting in real-time physiological data and/or target indicator data selected for the training, wherein:
the training prescription comprises predetermined training programs for a plurality of phases and a transition rule between the training programs for the plurality of phases; and
the training program comprises one or more training tasks that are predetermined to be executed, each of the one or more training tasks having a corresponding training scenario.

14. The system according to claim 13, wherein the method further comprises, prior to receiving a predetermined training configuration set in the training management module:
obtaining an assessment report and/or a training tool configured in the training management; and
configuring a corresponding training program based on the assessment report and/or the training tool of the subject.

15. The system according to claim 13, wherein the multi-parameter group biofeedback training system comprises:
a user management module configured to manage training configurations and training records of different subjects, wherein the user management module is configured to support operations on the training configuration and the training record of the subject, the operations comprising adding/deleting/modifying/querying, importing/exporting, batch importing, or batch deletion.

16. The system according to claim 15, wherein the multi-parameter group biofeedback training system further comprises:
a group management module configured to perform group management on different subjects based on a set need, wherein the group management module is configured to support the operations on the training configuration and the training record of the subject, the operations comprising adding/deleting/modifying/querying, importing/exporting, batch importing, or batch deletion.

17. The system according to claim 13, wherein the training management module is configured to support custom configuring a training configuration, wherein said custom configuring the training configuration comprises:
selecting target indicator data based on multi-parameter group biofeedback training;
selecting a training scenario compatible with the multi-parameter group biofeedback training, wherein types of the training scenario comprise any one or a combination of a VR scenario, an audio-video scenario, a virtual multi-person interactive animation scenario, a in virtual multi-person interactive game scenario, a multi-person interactive virtual-real fusion scenario, a screen-based 3D scenario, a visualization chart component, or an audio-video game resource scenario; and
performing an interactive setting between different scenarios based on a transition rule between different training programs in the training prescription that is pre-obtained, wherein the interactive setting comprises an ending manner setting for ending the training prescription, and wherein the transition rule comprises a transition condition setting for transitioning between training programs and a transition relationship network.

18. The system according to claim 17, wherein said presenting the training scenario to the subject based on the training or the training prescription to execute the training, and collecting in real-time the physiological data and/or the target indicator data selected for the training comprises:
in a physiological feedback training scenario, selecting the target indicator data by freely combining physiological channels.

19. The system according to claim 17, wherein the method further comprises:
generating a group biofeedback training report based on a target indicator data presentation manner pre-configured by the training management module, wherein the target indicator data presentation manner comprises target indicator data to be presented and a visualization chart, wherein:
said custom configuring the training configuration further comprises:
configuring the target indicator data presentation manner, wherein the visualization chart comprises, but is not limited to, one or more of a pie chart, a bar chart, or a Venn diagram.

20. A computer-readable storage medium, having a computer program stored thereon, wherein the computer program, when executed by a processor, implements a multi-parameter group biofeedback training method which is implemented based on a multi-parameter group biofeedback training system comprising a training management module, the method comprising:
obtaining a training program and a training prescription that are predetermined, wherein each of the training program and the training prescription is configured in training management, and wherein the training management module is configured to comprise a training scenario, the training program being a combination of individual trainings, and the training prescription being a combination of long-term, time-sequenced phased trainings or training programs; and
presenting the training scenario to a subject based on the training or the training prescription to execute the training, and collecting in real-time physiological data and/or target indicator data selected for the training, wherein:
the training prescription comprises predetermined training programs for a plurality of phases and a transition rule between the training programs for the plurality of phases; and
the training program comprises one or more training tasks that are predetermined to be executed, each of the one or more training tasks having a corresponding training scenario.
